(19) United States
(12) Patent Application Publication
(10) Pub. No.: US 2026/0201485 A1
(43) Pub. Date: Jul. 16, 2026

(54) METHOD OF PRODUCING REDUCED IRON

(71) Applicant: JFE STEEL CORPORATION, Chiyoda-ku, Tokyo (JP)

(72) Inventors: Yoshiko NAKAHARA, Chiyoda-ku, Tokyo (JP); Kota MORIYA, Chiyoda-ku, Tokyo (JP); Koki TERUI, Chiyoda-ku, Tokyo (JP); Taihei NOUCHI, Chiyoda-ku, Tokyo (JP)

(73) Assignee: JFE STEEL CORPORATION, Chiyoda-ku, Tokyo (JP)

(21) Appl. No.: 19/137,381

(22) PCT Filed: Dec. 22, 2023
(86) PCT No.: PCT/JP2023/046273

§ 371 Date: Jun. 10, 2025

(30) Foreign Application Priority Data
Dec. 23, 2022 (JP) 2022-207619

(51) Int. Cl. C21B 13/00; C21B 13/02

(52) U.S. Cl. CPC C21B 13/0073; C21B 13/02; C21B 2100/22; C21B 2100/26; C21B 2100/282; C21B 2100/44

(57) ABSTRACT

Provided is a method of producing reduced iron that can achieve both energy savings and a decrease in CO₂ emissions. The system is configured to circulate and reuse top gas with a blowing process, a reduction process, a distribution process, a synthesis process, and a reforming process, and the distribution ratio of the top gas in the distribution process is controlled according to consumed H₂/CO, which is a ratio of the amount of H₂ consumed to the amount of CO consumed in the reduction process.

### TECHNICAL FIELD

The present disclosure relates to a method of producing reduced iron.

### BACKGROUND

In recent years, there has been a strong demand for energy savings in steelworks against the backdrop of global environmental issues and fossil fuel depletion issues. The raw material of iron is mainly iron oxide, and a reduction process to reduce this iron oxide is essential in steelworks. The most widespread and common reduction process worldwide uses a blast furnace. In a blast furnace, coke or pulverized coal reacts with oxygen in hot blast (air heated to about 1200° C.) from a tuyere. This reaction produces CO and H₂ as reducing gases, which are used to reduce the iron ore and the like in the furnace. Recent improvements in blast furnace operation technology have decreased the reducing agent rate (the amount of coke and pulverized coal used per tonne of hot metal produced) to about 500 kg/t, and the reducing agent rate has already almost reached a lower limit. Therefore, no further significant decrease in the reducing agent rate is expected.

On the other hand, in regions where natural gas is produced, a vertical reduction furnace (hereinafter also referred to as a shaft furnace) is often used to produce reduced iron. In this method, the reduction furnace is charged with sintered ore, pellets, or other agglomerated iron ore as the iron oxide raw material (hereinafter also simply referred to as iron oxide). Reducing gas including CO and H₂ is then blown into the reduction furnace to reduce the iron oxide to produce reduced iron. In this method, natural gas or the like is used as the feed gas for the reducing gas. This feed gas is heated and reformed together with top gas in a reformer. This produces reducing gas. Here, the top gas is gas after the iron oxide is reduced in the reduction furnace, and is typically discharged from the top of the reduction furnace. The reducing gas is blown into the reduction furnace and reacts with iron oxide supplied from the top of the furnace. The iron oxide is then reduced to reduced iron. The reduced iron is then cooled in a region below where the reducing gas is blown into the reduction furnace, and then discharged from the bottom of the reduction furnace.

Further, as mentioned above, the top gas, which is gas after iron oxide is reduced, is discharged from the reduction furnace, for example, from the top of the furnace. After dust collection and cooling is applied to the top gas, some is fed to the reformer as raw material for reformed gas. Further, remaining top gas is used as fuel gas for the reformer. In this method, the top gas used as fuel gas for the reformer is normally discharged out of the system.

As such a reduced iron production process, for example, Patent Literature (PTL) 1 describes a method of producing reduced iron by reforming exhaust gas from a reduction furnace and natural gas in a reformer to produce reducing gas consisting mainly of CO and H₂, blowing the reducing gas into the reduction furnace, and reducing iron oxide in the reduction furnace.

Further, PTL 2 describes a method of producing reduced iron by reforming coke oven gas and CO₂-removed top gas from a reduction furnace to produce reducing gas, which is then blown into a reduction furnace.

### CITATION LIST

### Patent Literature

PTL 1: JP 2017-088912 A
PTL 2: JP 6190522 B2

### SUMMARY

### Technical Problem

The method described in PTL 1 uses externally supplied natural gas for the production of reducing gas. Therefore, although lower than that of blast furnaces, there is a problem that a certain amount of CO₂ emissions are unavoidable.

Further, the method described in PTL 2 uses coke oven gas or converter gas produced in a steelworks to produce reducing gas. Here, in an integrated steelworks, coke oven gas and converter gas are needed as fuel gas for downstream processes such as a heating furnace and an annealing furnace. Therefore, when coke oven gas or converter gas is diverted to the reduced iron production process, a fuel gas shortage is caused in the downstream processes. As a result, natural gas is supplied from outside to compensate for the shortage of fuel gas in the downstream processes. That is, even with the method described in PTL 2, energy savings and a decrease in CO₂ emissions could not both be achieved, and the problem remained.

In view of the circumstances described, it would be helpful to provide a method of producing reduced iron that can realize both energy savings and a decrease in CO₂ emissions.

### Solution to Problem

The inventors have conducted extensive research to realize both energy savings and a decrease in CO₂ emissions, and developed a system that circulates and reuses top gas.

That is, the inventors developed
a top gas circulation and reuse system (hereinafter also simply referred to a circulation system) comprising:
a blowing process of blowing reducing gas into a reduction furnace;
a reduction process, in the reduction furnace, of reducing iron oxide by the reducing gas to obtain reduced iron;
a synthesis process of synthesizing regenerative methane gas from top gas discharged from the reduction furnace and hydrogen gas; and
a reforming process of using the regenerative methane gas as feed gas to obtain reducing gas from the feed gas.

The inventors further investigated and made the following discoveries.
In order to stably carry out an operation that achieves both energy savings and a decrease in CO₂ emissions, it is important to maintain a healthy mass balance, in particular the mass balance of C (carbon), in the circulation system described above.
For this purpose, it is important to control the gas balance of the circulation system so that the ratio of the amount of H₂ to the amount of CO in the reducing gas supplied to the reduction furnace (hereinafter also referred to as H₂/CO of the reducing gas) is always maintained within a certain range without significant fluctuations. The H₂/CO of the reducing gas is the volume ratio under standard conditions (which may also be called the flow rate ratio under standard conditions). The same is true for consumed H₂/CO and the like, as described below.

There is an optimum energy range for the H₂/CO of the reducing gas, depending on furnace temperature and pressure in a direct reduction furnace and furnace size. Further, as examples of raw material for the reducing gas, in addition to natural gas used in a typical direct reduction furnace such as Midrex® (Midrex is a registered trademark in Japan, other countries, or both) and Hyl® (Hyl is a registered trademark in Japan, other countries, or both), coke oven gas, hydrocarbon, waste plastic, and biomass combustion gas may be used. When these gases are used, the H₂/CO of the reducing gas also changes to various values depending on composition. The direct reduction furnaces currently in operation are actually operating in a range of the H₂/CO of the reducing gas appropriate for their respective plants. However, the composition of top gas may change when the grade of iron oxide, the raw material of reduced iron, changes or when furnace temperature changes. In such cases, the H₂/CO of the reducing gas also changes, making it difficult to maintain a healthy mass balance in the circulation system described above.

Regarding the above points, the inventors made further studies and found that the following controls make it possible to maintain a healthy mass balance in the circulation system, that is, stably carry out an operation that achieves both energy savings and a decrease in CO₂ emissions during the production of reduced iron.
Top gas discharged from the reduction furnace is distributed into a first top gas and a second top gas.
The distribution ratio of the top gas in the distribution process is controlled according to the ratio of the amount of H₂ consumed to the amount of CO consumed in the reduction process, that is, consumed H₂/CO.

The present disclosure is based on these discoveries and further studies. Primary features of the present disclosure are as follows.

1. A method of producing reduced iron, the method comprising:
a charging process of charging iron oxide into a reduction furnace;
a blowing process of blowing reducing gas into the reduction furnace;
a reduction process, in the reduction furnace, of reducing the iron oxide by the reducing gas to obtain reduced iron;
a distribution process of distributing top gas discharged from the reduction furnace into a first top gas and a second top gas;
a synthesis process of synthesizing regenerative methane gas from the first top gas and hydrogen gas; and
a reforming process of using the regenerative methane gas and the second top gas as feed gas to obtain the reducing gas from the feed gas, wherein
a distribution ratio of the top gas in the distribution process is controlled according to consumed H₂/CO, a ratio of an amount of H₂ consumed to an amount of CO consumed in the reduction process.

2. The method of producing reduced iron according to 1, above, the method further comprising a separation process between the distribution process and the synthesis process, of separating the first top gas into carbon dioxide gas and remaining gas, wherein
the carbon dioxide gas is used as the first top gas in the synthesis process.

3. The method of producing reduced iron according to 1 or 2, above, wherein a distribution ratio, in %, of Y×100 to the first top gas in the distribution process is controlled in a range of Y₀×100±5%, Y₀×100 being a standard distribution ratio, in %,
where

$$when X \leq 1 , {Y}_{0} = 1 / ( 2 η )$$

$$when 1 < X < 4 η - 1 , {Y}_{0} = ( X + 1 ) / ( 4 η )$$

$$when X \geq 4 η - 1 , {Y}_{0} = 1$$

and
X is consumed H₂/CO
η×100 is CO₂ conversion rate, in %, from methane synthesis in the synthesis process
and when the range of Y₀×100±5% exceeds 100%, an upper limit of the range is 100%.

4. The method of producing reduced iron according to 3, above, wherein the distribution ratio, in %, of Y×100 to the first top gas in the distribution process is controlled to Y₀×100 being the standard distribution ratio, in %.

5. The method of producing reduced iron according to 3 or 4, above, wherein excess H₂ is recovered from a circulation system comprising the blowing process, the reduction process, the distribution process, the synthesis process, and the reforming process when the consumed H₂/CO<1.

6. The method of producing reduced iron according to 3 or 4, above, wherein H₂ is additionally supplied to a circulation system comprising the blowing process, the reduction process, the distribution process, the synthesis process, and the reforming process when the consumed H₂/CO>4η−1.

7. The method of producing reduced iron according to any one of 1 to 6, above, wherein at least one of de-dusting or dehydration of the top gas is carried out prior to the distribution process.

8. The method of producing reduced iron according to any one of 1 to 7, above, wherein dehydration of the regenerative methane gas is carried out prior to the reforming process.

### Advantageous Effect

According to the present disclosure, it is possible to stably carry out an operation that achieves both energy savings and a decrease in CO₂ emissions during the production of reduced iron.

### BRIEF DESCRIPTION OF THE DRAWINGS

In the accompanying drawings:

FIG. 1 is a diagram illustrating a conventional reduced iron production process;

FIG. 2 is a diagram illustrating an example of a reduced iron production process according to a method of producing reduced iron according to an embodiment of the present disclosure; and

FIG. 3 is a diagram illustrating the relationship between consumed H₂/CO:X and distribution ratio Y of a first top gas.

### DETAILED DESCRIPTION

The following is a description of a method of producing reduced iron according to an embodiment of the present disclosure, with reference to the drawings.

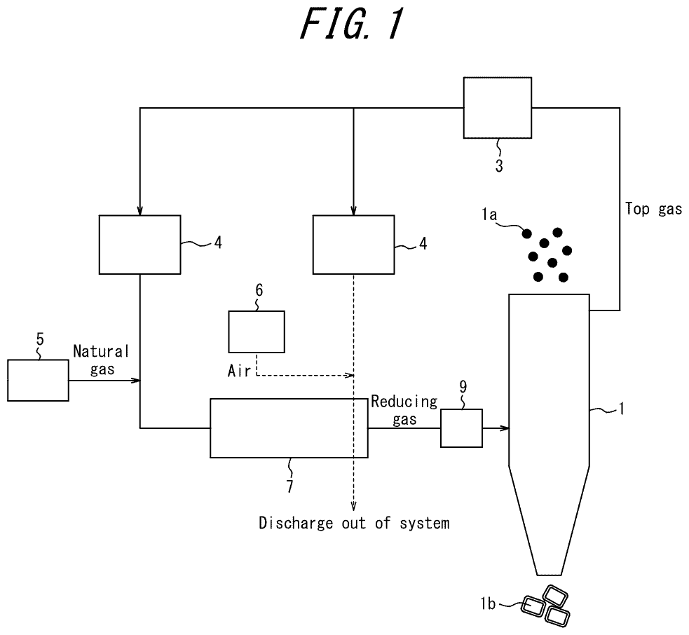

First, a conventional reduced iron production process (hereinafter also referred to as a conventional production process) is described. FIG. 1 is a schematic diagram illustrating an example of a conventional production process. In the drawing, reference sign 1 is a reduction furnace, 1a is iron oxide, 1b is reduced iron, 3 is a deduster, 4 is a dehydrator, 5 is a natural gas supply, 6 is an air supply, 7 is a reformer, and 9 is a reducing gas blowing device.

In the example of a conventional production process, iron oxide is charged from the top of the reduction furnace and gradually descends. There, iron oxide is reduced by blowing in high-temperature reducing gas from a central portion of the reduction furnace. The reduced iron is then discharged from the bottom of the reduction furnace. In this process, top gas containing mainly CO, CO₂, H₂, and H₂O is discharged from the top of the reduction furnace. The top gas is de-dusted by the deduster, and a portion is subjected to moisture adjustment and fed to the reformer as feed gas. Gas containing hydrocarbons, for example, natural gas from a natural gas supply, is supplied to the reformer along with the top gas that has been subjected to moisture adjustment. The supplied gas is then heated in the reformer. A reforming reaction then occurs, producing high-temperature reducing gas containing mainly CO and H₂. This reducing gas is then blown into the reduction furnace. Further, the remaining portion of the top gas is dehydrated and used as fuel for heating in the combustion chamber of the reformer, for example, by combustion with oxygen in the air. After combustion as fuel for heating, the top gas still containing CO₂ is normally discharged out of the system.

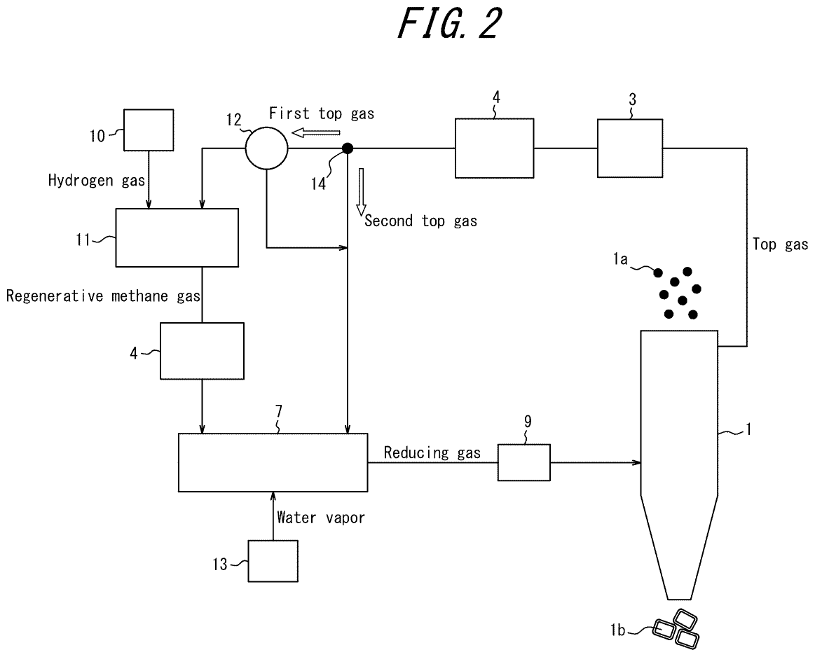

In contrast, in the method of producing reduced iron according to an embodiment of the present disclosure, as illustrated in FIG. 2 for example, the top gas discharged from the reduction furnace is distributed into first top gas and second top gas in a top gas distributor. Methane gas synthesized from the first top gas and hydrogen gas (hereinafter also referred to as regenerative methane gas) is used instead of hydrocarbon gas such as natural gas supplied from outside, as in the conventional process illustrated in FIG. 1. In the drawing, reference sign 10 is a hydrogen supply, 11 is a methane synthesizer, 12 is a CO₂ separator, 13 is a water vapor supply, and 14 is a top gas distributor. Here, the regenerative methane gas produced in the methane synthesizer 11 is supplied to the reformer 7 to be used as feed gas for the reducing gas.

The following is a description of each process of the method of producing reduced iron according to an embodiment of the present disclosure. The charging process, blowing process, and reduction process can be carried out in accordance with conventional methods, for example, in the same manner as in the conventional process described above, and therefore description is omitted here.

### Distribution Process

In the distribution process, for example, the top gas discharged from the reduction furnace is distributed into the first top gas and the second top gas in the top gas distributor. In this case, it is important to control a distribution ratio to the first top gas according to consumed H₂/CO, which is the ratio of the amount of H₂ consumed to the amount of CO consumed in the reduction process. The reasons for this are discussed below. Further, distribution of the top gas and a flow rate controller are not particularly limited and may follow a conventional method. For example, a mass flow controller or the like may be used.

### Separation Process

Between the distribution process described above and the synthesis process described below, a separation process may be further included, of separating the first top gas into carbon dioxide gas and remaining gas by, for example, a CO₂ separator. The method of CO₂ separation is not particularly limited, and various methods may be used, examples including chemical absorption, physical absorption, adsorption separation, membrane separation, deep cold separation, oxygen combustion, chemical loop combustion, and the like. Among these, in particular, chemical absorption methods, of which an amine absorption method is representative, and adsorption separation methods, of which a PSA method is representative, have long been used in chemical plants, industrial CO₂ production, and the like. The CO₂ concentration in the carbon dioxide gas is preferably 90 vol % or more. The CO₂ concentration in the carbon dioxide gas may be 100 vol %. Further, the remaining gas may, for example, be introduced into the reforming process described below, together with the second top gas.

### Synthesis Process

In the synthesis process, for example, regenerative methane gas is synthesized in a methane synthesizer from the first top gas distributed by the distribution process described above and hydrogen gas. Further, in the case of the separation process described above, carbon dioxide gas is suitable to be used as the first top gas in this process. CH₄ is synthesized from at least one of CO₂ or CO in the first top gas and H₂ according to the following methanation reaction equations in Expressions (i) and (ii).

$$\begin{matrix}{CO}_{2} + 4 {H}_{2} \to {CH}_{4} + 2 {H}_{2} O & ΔH = - 1 \; 65 kJ / mol\end{matrix} \qquad ( i )$$

$$\begin{matrix}CO + 3 {H}_{2} \to {CH}_{4} + {H}_{2} O & ΔH = - 2 \; 06 kJ / mol\end{matrix} \qquad ( ii )$$

For example, the first top gas and hydrogen gas supplied from outside the circulation system described above are introduced into the methane synthesizer. CH₄ is then synthesized in the methane synthesizer by at least one of the reactions in Expressions (i) and (ii). Conditions for CH₄ synthesis are not particularly limited, and a conventional method may be followed.

When the carbon dioxide gas obtained in the separation process described above is used, the composition of the first top gas is the same as that of the carbon dioxide gas. Further, when the separation process described above is not carried out, the composition of the first top gas is basically the same as the composition of the top gas. In such a case, the composition of the first top gas is CO: 5 vol % to 50 vol %, CO₂: 5 vol % to 30 vol %, H₂: 5 vol % to 80 vol %, H₂O: 0 vol % to 35 vol %, with the balance being: 0 vol % to 20 vol %. The composition of the second top gas, described below, is similar.

In addition to the first top gas, any other gas containing at least one of CO or CO₂ (hereinafter also referred to as “other gas”) may be used in the synthesis process. Examples of the other gas include by-product gas of a steelmaking process, specifically blast furnace gas (BFG) and coke oven gas (COG). Further, the other gas may be introduced into the separation process described above along with the first top gas and separated into carbon dioxide gas and remaining gas before supplying the carbon dioxide gas to the synthesis process.

Further, the source of hydrogen gas used in the synthesis process is not particularly limited and the hydrogen gas may be supplied and produced by any method. Methods of producing hydrogen gas include, for example, synthesis by electrolysis of water and by decomposition reactions of ammonia, hydrocarbons, and organic hydrides. However, when hydrocarbons or organic hydrides are used as a raw material, CO₂ is emitted in the hydrogen synthesis process. Therefore, from the viewpoint of further decreasing CO₂ emissions, synthesis by at least one of the electrolysis of water or decomposition of ammonia is preferred. Further, when hydrogen gas is produced by electrolysis of water, green hydrogen produced using electrical power from green energy sources such as solar, wind, and geothermal power may be used to decrease CO₂ emissions to zero. The H₂ concentration of the hydrogen gas is not particularly limited. The H₂ concentration of the hydrogen gas is preferably 90 vol % or more. The H₂ concentration of the hydrogen gas is more preferably 95 vol % or more. The H₂ concentration of the hydrogen gas may be 100 vol %.

Further, the amount of hydrogen gas supplied in the synthesis process (the amount of hydrogen gas supplied to the methane synthesizer) is preferably a stoichiometric amount relative to the amount of CO₂ contained in the first top gas, based on the methanation reaction equation in Expression (i).

In the synthesis of CH₄, a typically used methanation catalyst may be used. Specifically, a transition metal catalyst such as Fe, Ni, Co, Ru, or the like may be used. Among these, Ni catalysts are the most active. Further, Ni catalysts also have high heat resistance and are usable up to about 500° C. Therefore, Ni catalysts are particularly preferred. Further, iron ore may be used as a catalyst. Ores containing high crystallization water are particularly suitable for use as catalysts because the specific surface area increases when the water of crystallization is dehydrated.

The reactor of the methane synthesizer used in the synthesis process may be a fixed bed reactor, a fluidized bed reactor, an air flow bed reactor, or the like. Depending on the form of these reactors, the physical properties of the catalyst may be selected accordingly. Further, a heat exchanger may be disposed in the gas flow path downstream of the reactor to recover heat from the reaction heat (gas sensible heat) of the methanation reaction in each reactor. The recovered thermal energy may, for example, be used to heat the reduction furnace or the reformer.

Further, the methanation catalysts described above exhibit a stable high conversion rate during CO₂ methanation. However, there have been reported cases of catalyst poisoning during CO methanation due to C precipitation caused by the formation of C intermediates. Therefore, it is preferable to have a separation process between the distribution process and the synthesis process, where the first top gas is separated into carbon dioxide gas and remaining gas. In other words, the carbon dioxide gas separated from the first top gas in the separation process is used as the first top gas in the synthesis process.

When H₂O produced as a byproduct of methane synthesis is introduced into the reformer, there may be an excess of H₂O in the reforming process described below. Therefore, while considering the mass balance of the circulation system as a whole, it is preferable to dehydrate the regenerative methane gas using a dehydrator, as appropriate, prior to the reforming process described below.

The CH₄ concentration of the regenerative methane gas is not particularly limited. In regenerative methane gas from which H₂O is removed, that is, in regenerative methane gas after dehydration of the gas leaving the methane synthesizer by a dehydrator, the CH₄ concentration is preferably 80 vol % or more. The CH₄ concentration is more preferably 90 vol % or more. The CH₄ concentration in regenerative methane gas without H₂O may be 100 vol %.

### Reforming Process

In the reforming process, the regenerative methane gas and the second top gas are used as feed gas to obtain reducing gas from the feed gas. For example, the regenerative methane gas and the second top gas are introduced into the reformer, and the regenerative methane gas and the second top gas are heated in the reformer. Then, in the reformer, the reforming reactions of the following Expressions (iii) and (iv) are used to produce reducing gas containing CO and H₂. By supplying water vapor to the reformer, the reforming reaction represented in Expression (iv) proceeds.

$$\begin{matrix}{CH}_{4} + {CO}_{2} \to 2 CO + 2 {H}_{2} & Δ H = 247 kJ / mol\end{matrix} \qquad ( iii )$$

$$\begin{matrix}{CH}_{4} + {H}_{2} O \to CO + 3 {H}_{2} & ΔH = 206 kJ / mol\end{matrix} \qquad ( iv )$$

The gas composition of the reducing gas is, for example, CO: 1 vol % to 60 vol %, H₂: 40 vol % to 99 vol %, with the balance being 0 vol % to 30 vol %.

As indicated by the positive enthalpies of formation in Expressions (iii) and (iv), both reactions in Expressions (iii) and (iv) are endothermic reactions. Therefore, by using green energy sources such as solar power, wind, geothermal energy, and the like as an energy source during the reforming reaction, CO₂ emissions may in principle be decreased to zero.

The reducing gas is then introduced into the reduction furnace through a blowing process. For example, the reducing gas is introduced into the reduction furnace using a reducing gas blowing device. The iron oxide is then reduced by the reducing gas in the reduction furnace to obtain reduced iron. On the other hand, the reducing gas after being used for iron oxide reduction is discharged from the reduction furnace as the top gas.

Further, it is preferred that at least one of de-dusting or dehydration of the top gas is carried out prior to the distribution process. Any deduster may be used for de-dusting. Further, any dehydrator may be used for dehydration. The order of dedusting and dehydration is not particularly limited. In the example illustrated in FIG. 2, the top gas is dehydrated by the dehydrator after de-dusting by the deduster, and then the top gas is distributed into the first top gas and the second top gas.

### Top Gas Distribution Ratio in Distribution Process

In the method of producing reduced iron according to an embodiment of the present disclosure, it is important to control the distribution ratio of the top gas in the distribution process according to the consumed H₂/CO, which is the ratio of the amount of H₂ consumed to the amount of CO consumed in the reduction process (hereinafter also simply referred to as consumed H₂/CO).

Here, the consumed H₂/CO is calculated, for example, by the following expression.

$$consumed {H}_{2} / CO = ( [ amount of {H}_{2} contained in reducing gas blown into reduction furnace ( {Nm}^{3} / t ) ] - [ amount of {H}_{2} contained in top gas ( {Nm}^{3} / t ) ] ) / ( [ amount of CO contained in reducing gas blown into reduction furnace ( {Nm}^{3} / t ) ] - [ amount of CO contained in top gas ( {Nm}^{3} / t ) ] )$$

Here, Nm³/t is the basic unit per tonne of reduced iron (DRI) produced.

For example, the distribution ratio of Y×100(%) to the first top gas is controlled around the standard distribution ratio of Y₀×100(%), which is calculated according to X. For example, Y×100(%) is preferably controlled in the range of Y₀×100(%)±5%. Y×100(%) is more preferably in the range of Y₀×100(%)±3%. Y×100(%) is even more preferably in the range of Y₀×100(%)±1%. The distribution ratio of Y×100(%) to the first top gas is most preferably controlled to Y₀×100(%).

Here,

$$when X \leq 1 , {Y}_{0} = 1 / ( 2 η ) ,$$

$$when 1 < X < 4 η - 1 , {Y}_{0} = ( X + 1 ) / ( 4 η ) , and$$

$$when X \geq 4 η - 1 , {Y}_{0} = 1.$$

Further,
X is consumed H₂/CO, and
η×100 is CO₂ conversion rate from methane synthesis in the synthesis process (%).

Further, when the ranges of Y₀×100±5%, Y₀×100±3%, and Y₀×100±1% each exceed 100%, an upper limit of the ranges is 100%.

Y is defined by the following expression.

$$Y = [ amount of top gas distributed to first top gas in distribution process ( {Nm}^{3} / t ) ] \div [ amount of top gas introduced into distribution process ( {Nm}^{3} / t ) ]$$

The distribution ratio to the second top gas is also defined in the same way as in the above expression. The top gas is basically distributed into the first top gas and the second top gas, and therefore the distribution ratio to the second top gas is also appropriately controlled by appropriate control of the distribution ratio to the first top gas. Further, in this case, the distribution ratio to the second top gas is 1-Y. In addition, a portion of the top gas may be supplied to other loads depending on operating conditions and other factors, as long as the portion is 10 vol % or less of the top gas volume. The amount of the top gas supplied to other loads is not included in the amount of the top gas introduced into the distribution process.

Further, the CO₂ conversion rate η×100(%) from methane synthesis in the synthesis process is defined by the following expression.

$$[ {CO}_{2} conversion rate η \times 100 ( \% ) ] = ( 1 - [ amount of {CO}_{2} contained in regenerative methane gas emitted after synthesis of {CH}_{4} in synthesis process ( {Nm}^{3} / t ) ] \div [ amount of {CO}_{2} introduced into synthesis process ( {Nm}^{3} / t ) ] ) \times 100$$

The following describes the standard distribution ratio of Y₀×100(%). First, when iron oxide is reduced by a reducing gas that is a mixed gas of H₂ and CO, the consumption of H₂ and CO is determined by the equilibrium and reaction rate constants in Expressions (v) and (vi) below. For example, when the grade of the raw iron ore decreases, the activation energy of H₂ reduction increases, which is assumed to reduce the progression of Expression (vi). Thus, the amount of H₂ and the amount of CO consumed in reduction varies in various ways depending on operating conditions.

$$3 CO + {Fe}_{2} {O}_{3} \to 3 {CO}_{2} + 2 Fe \qquad ( v )$$

$$3 {H}_{2} + {Fe}_{2} {O}_{3} \to 3 {H}_{2} O + 2 Fe \qquad ( vi )$$

In the method of producing reduced iron according to an embodiment of the present disclosure, H₂ and CO equivalent to the amounts consumed by reduction are synthesized by reforming CH₄ to compensate for the consumption, thereby making it possible to keep the composition of the reducing gas blown into the reduction furnace constant. That is, in Expression (v), the amount of CO consumed by the reduction is equivalent to the amount of CO₂ produced by the reduction. A portion of this CO₂ is then converted to methane in the synthesis process as the first top gas, and then supplied to the reforming process. Further, the remaining CO₂ is supplied to the reforming process without going through the synthesis process as the second top gas. That is, focusing on mass balance for C overall, in the circulation system described above, the amount of CO consumed by the reduction of iron oxide in the reduction process is practically equivalent to the amount of CO produced in the reforming process.

Here, as indicated in Expressions (iii) and (iv), when CH₄ is reformed by CO₂, H₂ and CO are produced in a 1:1 ratio. On the other hand, when CH₄ is reformed by H₂O, H₂ and CO are produced in a ratio of 3:1. The distribution ratio to the first top gas is synonymous with the ratio of CO₂ in the top gas that is supplied to the synthesis process. The distribution ratio to the second top gas is synonymous with the ratio of CO₂ in the top gas that is supplied to the reforming process. In other words, the distribution ratio to the second top gas supplied to the reforming process can be said to represent the ratio of the reforming reaction due to CO₂ in Expression (iii). Therefore, the distribution ratio of the top gas is an important factor in maintaining a healthy mass balance in the circulation system, in particular the mass balance of C overall.

When the total carbon equivalent of CO₂ contained in the top gas introduced into the distribution process is set to 1, the carbon equivalent of CH₄ in the regenerative methane gas synthesized in the synthesis process is expressed as Yη, which is the distribution ratio Y of the first top gas multiplied by the CO₂ conversion ratio n from methane synthesis in the synthesis process.

Further, the CO₂ introduced into the reforming process is the sum of the CO₂ contained in the second top gas and the CO₂ remaining in the first top gas that is not synthesized into methane in the synthesis process. Therefore, when the total carbon equivalent of CO₂ contained in the top gas introduced into the distribution process is set to 1, the carbon equivalent of CO₂ introduced into the reforming process is represented by the following expression.

$$1 - Y + Y ( 1 - η ) = 1 - Y η$$

Then, for example, when X=1, that is, the ratio of the amount of H₂ and CO consumed in the reduction process is 1:1, 100% progress of the reaction in Expression (iii) produces a reducing gas with H₂:CO=1:1, which can compensate for H₂ and CO equivalent to the amounts consumed in the reduction process. In this case, it is necessary to supply the reforming process with an amount of CO₂ equivalent to the CH₄ contained in the regenerative methane gas.

$$Y η : 1 - Y η = 1 : 1$$

Transforming this,

$$Y = 1 / ( 2 η ) .$$

$$That is , when X = 1 , {Y}_{0} = 1 / ( 2 η ) .$$

Even when X<1, that is, the amount of CO consumed in the reduction process is greater than the amount of H₂, it is optimal to allow 100% of the reaction in Expression (iii) to proceed in order to maintain the mass balance. Therefore, Y₀=1/(2η) in this case as well.

On the other hand, as the value of X increases, the ratio of the H₂O reforming reaction in Expression (iv) needs to be increased. In other words, it is necessary to increase the proportion of CO₂ introduced into the synthesis process with respect to the CO₂ contained in the top gas by increasing the distribution ratio Y to the first top gas in the distribution process.

Here, when Y=1,
of the reforming reactions in the reforming process, the ratio of the CO₂ reforming reaction in Expression (iii) is represented by

$$1 - η ( \times 100 ( \% ) ) .$$

The ratio of the H₂O reforming reaction in Expression (iv) is represented by

$$η - ( 1 - η ) = 2 η - 1 ( \times 100 ( \% ) ) .$$

Therefore, the H₂/CO ratio of the reducing gas obtained in the reforming process is represented by the following expression.

$$\{ 2 \times ( 1 - η ) + 3 ( 2 η - 1 ) \} / \{ 2 \times ( 1 - η ) + 2 η - 1 \} = 4 η - 1$$

Therefore, when X=4η−1, Y=1, that is, all of the top gas is distributed as the first top gas in the distribution process, and the reforming reaction in Expression (iv) proceeding as much as possible according to n is optimal to maintain the mass balance. Therefore, Y₀=1 in this case.

Even when X>4η−1, that is, when the consumed H₂/CO in the reduction process exceeds 4η−1, it is optimal to allow the reaction in Expression (iv) to proceed as much as possible in order to maintain the mass balance. Therefore, Y₀=1 in this case.

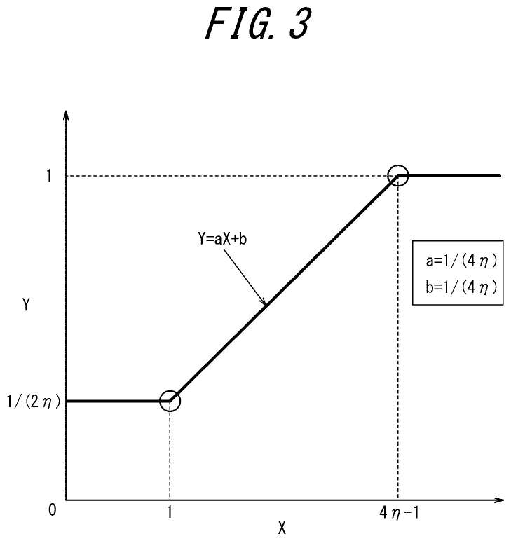

Further, when 1<X<4η−1, the reference distribution ratio Y₀ to the first top gas is, as illustrated in FIG. 3, a straight line Y₀=aX+b connecting two points with the threshold values Y₀=1/(2η) when X=1 and Y₀=1 when X=4η−1. Here, a and b are as follows.

$$a = 1 / ( 4 η )$$

$$b = 1 / ( 4 η )$$

$$That is , when 1 < X < 4 η - 1 , {Y}_{0} = ( X + 1 ) / ( 4 η ) .$$

The distribution ratio of Y×100(%) to the first top gas is controlled around the standard distribution ratio of Y₀×100(%), which is calculated according to X. For example, Y×100(%) is preferably controlled in the range of Y₀×100(%)±5%. Y×100(%) is more preferably in the range of Y₀×100(%)±3%. Y×100(%) is even more preferably in the range of Y₀×100(%)±1%. The distribution ratio of Y×100(%) to the first top gas is most preferably controlled to Y₀×100(%). This allows the gas balance of the circulation system to be controlled by maintaining the H₂/CO of the reducing gas within a certain range without significant fluctuations.

When X<1, even when the reaction in Expression (iii) proceeds 100%, then an excess of H₂ (hereinafter also referred to as excess H₂) is generated in the circulation system. Therefore, in this case, excess H₂ is preferably recovered from the circulation system. The process for recovering excess H₂ may be, for example, immediately before the reforming process or after the reforming process and before the blowing process. The amount of excess H₂ recovered is preferably determined so that the amount of H₂ contained in the reducing gas blown into the reduction furnace is kept constant and the reaction in Expression (iii) proceeds 100%. Therefore, in view of the above, the process of recovering excess H₂ is preferably after the reforming process and before the blowing process.

Further, in the case of X>4η−1, even when the reaction in Expression (iv) proceeds 100%, there will be a lack of H₂ in the circulation system. Therefore, in this case, additional H₂ is preferably supplied to the circulation system. The amount of additional H₂ supplied is preferably determined so that the amount of H₂ in the reducing gas blown into the reduction furnace is kept constant. Therefore, the process of additionally supplying H₂ is preferably, for example, immediately before the reforming process or after the reforming process and before the blowing process.

When the distribution ratio of Y to the first top gas does not match the standard distribution ratio of Y₀, the consumed H₂/CO and the H₂/CO of the reducing gas obtained in the reforming process do not exactly match. However, as mentioned above, it is acceptable when the distribution ratio of Y×100(%) to the first top gas is within the range of Y₀×100±5%. In this case, for example, H₂ is preferably supplied or recovered as appropriate to keep the amount of H₂ in the reducing gas constant.

Here, we also focus on the mass balance of the CO₂ cycle. That is, CO and H₂ are assumed to be unchanged before and after the synthesis process and the reforming processes, and therefore when the methanation reaction of CO according to Expression (ii) occurs in the synthesis process, the H₂/CO of the reducing gas changes. Therefore, as described above, it is preferable to have a separation process to separate the first top gas into carbon dioxide gas and remaining gas, and to use the carbon dioxide gas obtained in the separation process as the first top gas in the synthesis process. Further, a higher CO₂ concentration in the carbon dioxide gas is preferred, preferably 90 vol % or more. The CO₂ concentration in the carbon dioxide gas is optimally 100 vol %. When methanation of CO occurs in the synthesis process, for example, H₂ may be supplied or recovered as appropriate to keep the amount of H₂ in the reducing gas constant.

The distribution ratio to the first top gas in the distribution process may be constant or may be changed at any time. For example, the components of the reducing gas and the top gas can be measured and the distribution ratio to the first top gas in the distribution process can be changed according to the variation of X: consumed H₂/CO. Further, the distribution ratio to the first top gas at the time of apparatus startup may be determined, for example, from past operation history, and then the distribution ratio to the first top gas may be controlled as appropriate according to the variation of X: consumed H₂/CO.

Further, in the method of producing reduced iron according to an embodiment of the present disclosure, the flow rate of the reducing gas supplied to the reduction furnace is preferably 1500 Nm³/t or more. The flow rate is preferably 3500 Nm³/t or less. That is, when the flow rate of the reducing gas supplied to the reduction furnace is too low, production decreases and product properties degrade. On the other hand, when the flow rate of the reducing gas supplied to the reduction furnace is excessive, gas flow resistance in the reduction furnace increases and the raw material (pellet) in the reduction furnace does not descend. Therefore, the flow rate of the reducing gas supplied to the reduction furnace is preferably 1500 Nm³/t or more. The flow rate is preferably 3500 Nm³/t or less.

Further, the iron oxide raw material used in the method of producing reduced iron according to an embodiment of the present disclosure is, for example, iron ore. Examples include lumped iron ore (lump ore), pellets (powdered iron ore hardened into a spherical shape), and the like. The grade of iron ore used as iron oxide raw material, that is, the iron content, is not particularly limited. From the perspective of reduction in a shaft furnace, the iron content is typically preferably 65 mass % or more. However, in recent years, prices are expected to soar due to the depletion of high-grade iron ore from South America and other sources. Therefore, low-grade iron ore that has an iron content of 63 mass % or less is preferably used as required. Such low-grade iron ore is inexpensive and abundant, and places of origin include, for example, Australia.

In addition, the method of producing reduced iron according to an embodiment of the present disclosure describes, among other things, the use of a shaft furnace as a direct reduction iron-making process. However, the type of reduction furnace is not limited to this, and may be a fluidized bed, a rotary kiln, a rotary hearth furnace (RHF), or the like. Shaft furnaces are preferred as reduction furnaces because of their high production efficiency, ratio of utilization and operational stability. Further, the majority of direct reduction furnaces in operation worldwide are Midrex® and Hyl®, which are shaft furnace systems.

### EXAMPLES

Examples of the present disclosure are described below.

In the circulation system illustrated in FIG. 2, reduced iron was produced according to the conditions listed in Table 1. Under all conditions, the flow rate of the reducing gas supplied to the reduction furnace was 2200 Nm³/t, and the operation period was 28 days. In the distribution process, all the introduced top gas was distributed between the first top gas and the second top gas, and was not supplied to other loads. In the separation process, the first top gas was separated into carbon dioxide gas (CO₂ concentration: 99 vol % or more) and remaining gas, and the carbon dioxide gas was used as the first top gas in the synthesis process. Further, the remaining gas was introduced into the reforming process along with the second top gas. Both the recovery of excess H₂ and the additional supply of H₂ were carried out after the reforming process and before the blowing process. Conditions other than those described above and in Table 1 were in accordance with a conventional method. Further, the flow rates of the reducing gas and the top gas in Table 1 are rounded to the nearest integer. Therefore, the total flow rate of the reducing gas may not match the total amount of H₂ and CO in the reducing gas. The same is true for the top gas.

TABLE 1

| | | | | | | | | | Methane |
|---|---|---|---|---|---|---|---|---|---|
| | Reducing gas | | | | Top gas | | | | synthesis |
| | | H₂ | Amount | | | H₂ | Amount | | CO₂ |
| | Flow rate | amount | of CO | | Flow rate | amount | of CO | | conversion |
| No. | [Nm³/t] | [Nm³/t] | [Nm³/t] | H₂/CO | [Nm³/t] | [Nm³/t] | [Nm³/t] | H₂/CO | rate η |
| 1 | 2200 | 200 | 2000 | 0.10 | 1646 | 108 | 1538 | 0.07 | 0.92 |
| 2 | | 508 | 1692 | 0.30 | 1646 | 319 | 1327 | 0.24 | 0.92 |
| 3 | | 906 | 1294 | 0.70 | 1704 | 671 | 1033 | 0.65 | 0.90 |
| 4 | | 1100 | 1100 | 1.00 | 1679 | 840 | 840 | 1.00 | 0.92 |
| 5 | | 1320 | 880 | 1.50 | 1646 | 1013 | 633 | 1.60 | 0.91 |
| 6 | | 1467 | 733 | 2.00 | 1704 | 1171 | 532 | 2.20 | 0.92 |
| 7 | | 1533 | 667 | 2.30 | 1704 | 1231 | 473 | 2.60 | 0.90 |
| 8 | | 1650 | 550 | 3.00 | 1646 | 1263 | 383 | 3.30 | 0.78 |
| 9 | | 1711 | 489 | 3.50 | 1646 | 1295 | 350 | 3.70 | 0.90 |
| 10 | | 1833 | 367 | 5.00 | 1679 | 1429 | 251 | 5.70 | 0.85 |
| 11 | | 1980 | 220 | 9.00 | 1646 | 1519 | 127 | 12.00 | 0.89 |
| 12 | | 1467 | 733 | 2.00 | 1704 | 1171 | 532 | 2.20 | 0.92 |
| 13 | | 1467 | 733 | 2.00 | 1704 | 1171 | 532 | 2.20 | 0.93 |
| 14 | | 1467 | 733 | 2.00 | 1704 | 1171 | 532 | 2.20 | 0.90 |
| 15 | | 1467 | 733 | 2.00 | 1704 | 1171 | 532 | 2.20 | 0.90 |
| 16 | | 1467 | 733 | 2.00 | 1704 | 1171 | 532 | 2.20 | 0.91 |
| 17 | | 1467 | 733 | 2.00 | 1704 | 1171 | 532 | 2.20 | 0.92 |
| 18 | | 1467 | 733 | 2.00 | 1704 | 1171 | 532 | 2.20 | 0.92 |

| | Distribution process | | | | | | | | |
|---|---|---|---|---|---|---|---|---|---|
| | Control of | | | | | | | | |
| | distribution | | | | Recovery of | | Additional supply | | |
| | ratio | | | | excess H₂ | | of H₂ | | |
| | according to | | | | Yes | Volume | Yes | Additional | |
| | consumed | | | | or | recovered | or | supply | |
| No. | H₂/CO ratio | X | Y₀ | Y | no | [Nm³/t] | no | [Nm³/t] | Remarks |
| 1 | Yes | 0.20 | 0.54 | 0.54 | Yes | 370 | No | — | Example |
| 2 | Yes | 0.52 | 0.54 | 0.54 | Yes | 176 | No | — | Example |
| 3 | Yes | 0.90 | 0.56 | 0.56 | Yes | 27 | No | — | Example |
| 4 | Yes | 1.00 | 0.54 | 0.54 | No | — | No | — | Example |
| 5 | Yes | 1.24 | 0.62 | 0.62 | No | — | No | — | Example |
| 6 | Yes | 1.47 | 0.67 | 0.67 | No | — | No | — | Example |
| 7 | Yes | 1.57 | 0.71 | 0.71 | No | — | No | — | Example |
| 8 | Yes | 2.31 | 1.00 | 1.00 | No | — | No | 32 | Example |
| 9 | Yes | 3.00 | 1.00 | 1.00 | No | — | No | 55 | Example |
| 10 | Yes | 3.49 | 1.00 | 1.00 | No | — | Yes | 126 | Example |
| 11 | Yes | 4.93 | 1.00 | 1.00 | No | — | Yes | 222 | Example |
| 12 | No | 1.47 | — | — | No | — | No | — | Comparative |
| | | | | | | | | | Example |
| 13 | Yes | 1.47 | 0.66 | 0.70 | No | 27 | No | — | Example |
| 14 | Yes | 1.47 | 0.69 | 0.70 | No | 10 | No | — | Example |
| 15 | Yes | 1.47 | 0.69 | 0.68 | No | — | No | 4 | Example |
| 16 | Yes | 1.47 | 0.68 | 0.66 | No | — | No | 14 | Example |
| 17 | Yes | 1.47 | 0.67 | 0.63 | No | — | No | 30 | Example |
| 18 | Yes | 1.47 | 0.67 | 0.62 | No | — | No | 38 | Example |

In all of the Examples where the distribution ratio of the top gas in the distribution process was controlled according to the consumed H₂/CO, the circulation system illustrated in FIG. 2, which is extremely advantageous in realizing energy savings, that is, the system in which the top gas is circulated and reused, was stably operated with a healthy mass balance over the entire 28-day operation period. Further, CO₂ emissions from the circulation system could be decreased to zero.

For comparison, as No. 12, the production of reduced iron was carried out under the same conditions as No. 6, except that all of the top gas was introduced into the synthesis process without controlling the distribution ratio of the top gas. Partway through the operation period, it became impossible to maintain stable operation with a healthy mass balance, and operation had to be suspended.

### REFERENCE SIGNS LIST

1 reduction furnace
1a iron oxide
1b reduced iron
3 deduster
4 dehydrator
5 natural gas supply
6 air supply
7 reformer
9 reducing gas blowing device
10 hydrogen supply
11 methane synthesizer
12 CO₂ separator
13 water vapor supply
14 top gas distributor

1. A method of producing reduced iron, the method comprising:
a charging process of charging iron oxide into a reduction furnace;
a blowing process of blowing reducing gas into the reduction furnace;
a reduction process, in the reduction furnace, of reducing the iron oxide by the reducing gas to obtain reduced iron;
a distribution process of distributing top gas discharged from the reduction furnace into a first top gas and a second top gas;
a synthesis process of synthesizing regenerative methane gas from the first top gas and hydrogen gas; and
a reforming process of using the regenerative methane gas and the second top gas as feed gas to obtain the reducing gas from the feed gas, wherein
a distribution ratio of the top gas in the distribution process is controlled according to consumed H₂/CO, a ratio of an amount of H₂ consumed to an amount of CO consumed in the reduction process.

2. The method of producing reduced iron according to claim 1, the method further comprising a separation process between the distribution process and the synthesis process, of separating the first top gas into carbon dioxide gas and remaining gas, wherein
the carbon dioxide gas is used as the first top gas in the synthesis process.

3. The method of producing reduced iron according to claim 1, wherein a distribution ratio, in %, of Y×100 to the first top gas in the distribution process is controlled in a range of Y₀×100±5%, Y₀×100 being a standard distribution ratio, in %,
where
$$when X \leq 1 , {Y}_{0} = 1 / ( 2 η )$$
$$when 1 < X < 4 η - 1 , {Y}_{0} = ( X + 1 ) / ( 4 η )$$
$$when X \geq 4 η - 1 , {Y}_{0} = 1$$
and
X is consumed H₂/CO
η×100 is CO₂ conversion rate, in %, from methane synthesis in the synthesis process
and when the range of Y₀×100±5% exceeds 100%, an upper limit of the range is 100%.

4. The method of producing reduced iron according to claim 3, wherein the distribution ratio, in %, of Y×100 to the first top gas in the distribution process is controlled to Y₀×100 being the standard distribution ratio, in %.

5. The method of producing reduced iron according to claim 3, wherein excess H₂ is recovered from a circulation system comprising the blowing process, the reduction process, the distribution process, the synthesis process, and the reforming process when the consumed H₂/CO<1.

6. The method of producing reduced iron according to claim 4, wherein excess H₂ is recovered from a circulation system comprising the blowing process, the reduction process, the distribution process, the synthesis process, and the reforming process when the consumed H₂/CO<1.

7. The method of producing reduced iron according to claim 3, wherein H₂ is additionally supplied to a circulation system comprising the blowing process, the reduction process, the distribution process, the synthesis process, and the reforming process when the consumed H₂/CO>4η−1.

8. The method of producing reduced iron according to claim 4, wherein H₂ is additionally supplied to a circulation system comprising the blowing process, the reduction process, the distribution process, the synthesis process, and the reforming process when the consumed H₂/CO>4η−1.

9. The method of producing reduced iron according to claim 1, wherein at least one of de-dusting or dehydration of the top gas is carried out prior to the distribution process.

10. The method of producing reduced iron according to claim 3, wherein at least one of de-dusting or dehydration of the top gas is carried out prior to the distribution process.

11. The method of producing reduced iron according to claim 1, wherein dehydration of the regenerative methane gas is carried out prior to the reforming process.

12. The method of producing reduced iron according to claim 3, wherein dehydration of the regenerative methane gas is carried out prior to the reforming process.

13. The method of producing reduced iron according to claim 2, wherein a distribution ratio, in %, of Y×100 to the first top gas in the distribution process is controlled in a range of Y₀×100±5%, Y₀×100 being a standard distribution ratio, in %,
where
$$when X \leq 1 , {Y}_{0} = 1 / ( 2 η )$$
$$when 1 < X < 4 η - 1 , {Y}_{0} = ( X + 1 ) / ( 4 η )$$
$$when X \geq 4 η - 1 , {Y}_{0} = 1$$
and
X is consumed H₂/CO
η×100 is CO₂ conversion rate, in %, from methane synthesis in the synthesis process
and when the range of Y₀×100±5% exceeds 100%, an upper limit of the range is 100%.

14. The method of producing reduced iron according to claim 13, wherein the distribution ratio, in %, of Y×100 to the first top gas in the distribution process is controlled to Y₀×100 being the standard distribution ratio, in %.

15. The method of producing reduced iron according to claim 13, wherein excess H₂ is recovered from a circulation system comprising the blowing process, the reduction process, the distribution process, the synthesis process, and the reforming process when the consumed H₂/CO<1.

16. The method of producing reduced iron according to claim 14, wherein excess H₂ is recovered from a circulation system comprising the blowing process, the reduction process, the distribution process, the synthesis process, and the reforming process when the consumed H₂/CO<1.

17. The method of producing reduced iron according to claim 13, wherein H₂ is additionally supplied to a circulation system comprising the blowing process, the reduction process, the distribution process, the synthesis process, and the reforming process when the consumed H₂/CO>4η−1.

18. The method of producing reduced iron according to claim 14, wherein H₂ is additionally supplied to a circulation system comprising the blowing process, the reduction process, the distribution process, the synthesis process, and the reforming process when the consumed H₂/CO>4η−1.

19. The method of producing reduced iron according to claim 2, wherein at least one of de-dusting or dehydration of the top gas is carried out prior to the distribution process.

20. The method of producing reduced iron according to claim 2, wherein dehydration of the regenerative methane gas is carried out prior to the reforming process.
